(19) United States
(12) Patent Application Publication
(10) Pub. No.: US 2020/0297881 A1
(43) Pub. Date: Sep. 24, 2020

(54) DEVICE, SYSTEM, AND METHOD FOR ANTIMICROBIAL TREATMENT, METHOD FOR PRODUCING THE DEVICE, AND COMPUTER PROGRAM

(71) Applicant: LEIBNIZ-INSTITUT FUER PLASMAFORSCHUNG UND TECHNOLOGIE E. V., Greifswald (DE)

(72) Inventors: Klaus-Dieter WELTMANN, Binz (DE); Thomas VON WOEDTKE, Sundhagen (DE); Manfred STIEBER, Greifswald (DE); Stefan HORN, Loissin (DE)

(73) Assignee: LEIBNIZ-INSTITUT FUER PLASMAFORSCHUNG UND TECHNOLOGIE E. V., Greifswald (DE)

(21) Appl. No.: 16/085,580

(22) PCT Filed: Mar. 16, 2017
(86) PCT No.: PCT/EP2017/056300

§ 371 Date: Sep. 16, 2018

(30) Foreign Application Priority Data
Mar. 16, 2016 (DE) 10 2016 104 933.6

(51) Int. Cl. A61L 2/14; A61B 18/14

(52) U.S. Cl. CPC A61L 2/14; A61B 18/1482; A61B 2018/00083; A61B 18/1492; A61B 18/1487

(57) ABSTRACT

The invention relates to a device, a system and a method for antimicrobial treatment during the performance of operations in bodies, a method for manufacturing the device as well as a computer program.

There is provided a device (11) for antimicrobial treatment during the performance of operations, in particular minimally invasive operations, in bodies. Said device comprises a base body (1) for partial introduction into a body and, furthermore, at least one plasma source (12) arranged in at least one portion of the base body (1). The plasma source (12) has at least one high-voltage electrode (9) which is at least partially covered and in particular completely covered with a dielectric (10), which high-voltage electrode is set up to produce a plasma by means of dielectric barrier discharge when an electrical voltage is applied and in conjunction with a second electrode.

The invention relates to a device, a system and a method for antimicrobial treatment during the performance of operations in bodies, a method for manufacturing the device as well as a computer program.

Transcutaneous access points to the interior of the body are located via surgically positioned, artificial openings in the body surface for minimally invasive surgery. Experience has shown that access points of this nature are characterized by a relatively high risk of infection so that appropriate disinfecting measures which prevent infections are required. The situation is also similar for endoscopic applications in body cavities.

Since disinfecting agents can cause allergic reactions, on the one hand, and are only suitable to a limited extent as a prophylaxis against infections such as e.g. methicillin-resistant Staphylococcus aureus (MRSA) infections, on the other hand, the application of an antimicrobially effective plasma treatment in this region is advantageous, especially since no resistance to the plasma effect is known to date. In particular, cold atmospheric-pressure plasma is well suited to this.

WO 2015 181 325 A1 or respectively DE 10 2014 107 554 A1 describes a device for biologically decontaminating percutaneous access points or stomata with a plasma generator. Said plasma generator has a curved treatment surface which is set up to surround the percutaneous access point or the stomata and can be laid against said percutaneous access point or stomata. The device can, for example, be designed to be forceps with movable forceps jaws, on the inner sides of which treatment surfaces are arranged.

Consequently, an instrument for biologically decontaminating (degerming, disinfecting, sterilizing) percutaneous (transcutaneous) access points as well as skin areas in the region of transcutaneous access points by means of a cold atmospheric-pressure plasma is described. The described solutions for the antimicrobially effective plasma treatment of percutaneous access points are indeed to be advantageously used, especially in view of the effectiveness of the biological decontamination, but require the use of an external plasma source as an additional instrument for the plasma treatment.

It is the object of the invention to provide a device, a system and a method for antimicrobial treatment during the performance of operations in bodies, with which a plasma for antimicrobial treatment of bodies can be produced in a particularly simple and inexpensive manner. Furthermore, it is an object of the invention to provide a method for manufacturing the device as well as a computer program for performing the method for antimicrobial treatment.

The object is achieved by the device for antimicrobial treatment during the performance of operations in bodies according to claim 1, by the method for manufacturing the device according to claim 10, by the system for antimicrobial treatment during the performance of operations in bodies according to claim 11, by the method for antimicrobial treatment during the performance of operations in bodies according to claim 13 as well as by the computer program according to claim 15. Configurations of the device are indicated in the subordinate claims 2-9, a configuration of the system is indicated in subordinate claim 12 and a configuration of the method for antimicrobial treatment is indicated in subordinate claim 14.

A first aspect of the invention is a device for antimicrobial treatment during the performance of operations, in particular minimally invasive operations, in bodies. Said device comprises a base body for partial introduction into a body and, furthermore, at least one plasma source arranged in at least one portion of the base body. The plasma source has at least one high-voltage electrode which is at least partially covered and in particular completely covered with a dielectric, which high-voltage electrode is set up to produce a plasma by means of dielectric barrier discharge when an electrical voltage is applied and in conjunction with a second electrode.

In accordance with the invention, the dielectric is in particular a solid which only conducts weakly electrically or respectively which does not conduct electrically, and which consists of a non-metallic material. In particular, it can be a plastic, for example polyethylene or PTFE, or a ceramic material such as, for instance, steatite, aluminum oxide or silicate.

The high-voltage electrode is an electrical conductor which is suitable for applying a high voltage. Typically, it is manufactured from a metallic material. In this case, it can be a solid metal body or can be designed as a mesh, fabric or similar, for example as a metal gauze. A design as a wire is also conceivable. Such a wire can, in this case, be wound up or can have a meandering design. In particular, the high-voltage electrode is applied to the base body as a metal layer which is at least closed in certain areas. It can be arranged circumferentially directly or indirectly on the base body in the angular direction with respect to the longitudinal axis of the base body.

The antimicrobial treatment is a disinfecting, sterilizing or respectively degerming treatment. Accordingly, the device is suitable, e.g. for biologically decontaminating, degerming, disinfecting or respectively sterilizing percutaneous or respectively transcutaneous access points as well as skin areas in the region of such access points.

The plasma which can be produced with the device is in particular a low-temperature plasma, that is to say a cold atmospheric-pressure plasma. The underlying process is dark electrical discharge or respectively dielectric barrier discharge. Consequently, the device which can be used for performing surgical operations itself becomes the plasma source. The base body of the device is in particular an elongated base body.

The second electrode required for producing the plasma is an earthed counter-electrode which is not necessarily part of the device. For the purposes of producing the plasma, it is to be arranged at a distance from the high-voltage electrode, wherein the dielectric is to typically be arranged between the electrodes.

In particular, the electrical voltage means an alternating voltage. This typically has a high frequency in the range of 1 kHz to 1000 kHz. The electrical voltage is in particular a high voltage in the kilovolt range.

A body in accordance with the invention can be a human or animal body or a part thereof. Furthermore, the body can be an open or closed cavity, for example a tank, or a space at least partially filled with a liquid, a gas or a solid, wherein this solid can, for example, also be biological material.

If the body is a human or animal body or a part thereof, the introduction can take place through the skin (transcutaneously or percutaneously) or through existing body openings.

A plasma source is substantially configured by the first high-voltage electrode which can produce a plasma if an earthed counter-electrode is present and does not comprise the voltage source here.

A high-voltage electrode for generating a dielectric barrier discharge (DBD) for the purpose of producing a plasma, in particular a low-temperature plasma, is applied by an electrically conducting coating on the surface of the base body of a minimally invasive instrument or respectively endoscope. This is combined with a dielectrical coating. As a result, the instrument itself can, following the application of a suitable high voltage to the electrode, be used as a plasma source for an antimicrobially effective plasma treatment in the contact region with the body tissue, in particular in the region of the edge of the wound at the inlet or respectively outlet opening, wherein the surrounding or respectively adjacent body tissue can function as an earthed counter-electrode. Likewise, openings of any bodies can be treated with plasma as described.

In other words, a device for performing a minimally invasive operation in a body is provided, which comprises an elongated base body and at least one plasma source. The plasma source is arranged in at least one portion of the base body and has at least one high-voltage electrode which is at least partially covered and preferably completely covered with a dielectric, which high-voltage electrode can produce a plasma based on a dielectric barrier discharge when an electrical voltage is applied in conjunction with a second electrode.

Consequently, an antimicrobial, plasma-supported treatment in the region of transcutaneous access points towards the interior of the body or access points to body cavities is made possible locally.

The purpose of such a device is to inactivate microorganisms at the inlet opening and, if applicable, in the interior of the body in the area surrounding the introduced instrument, possibly during the biological decontamination of transcutaneous access points or body cavities on humans and animals, and to stimulate the healing of the wound at the skin opening following removal of the instrument.

Thanks to the invention, the risk of infection in the case of minimally invasive surgical operations or respectively endoscopic examinations is successfully minimized in the region of the transcutaneous access points or respectively stomata, a substantial advantage with respect to the known solutions being that the instrument (minimally invasive instrument or respectively endoscope) can itself be used to produce plasma and, consequently, no additional, external plasma source is required. This is achieved by the described modification of the surface of the instrument base body.

In one configuration of the device, a spacer element for producing a distance between the plasma source and body tissue is arranged on the side of the dielectric facing away from the high-voltage electrode in at least one portion of the base body.

In one configuration, a spacer element for producing a distance between the plasma source and body tissue is arranged in a portion of the base body.

The spacer element is in particular arranged circumferentially around the base body in the angular direction. In this case, multiple portions of the base body can have a spacer element.

The spacer element is in particular not electrically conducting. It can in this case be manufactured from one of the described materials, which can also make up the dielectric. It can also comprise at least a part of the dielectric or rest thereon.

The spacer element is in particular mounted on the outside of the dielectric as a cover or respectively coating, so that it is set up to observe a minimum distance between the electrode or respectively dielectric and any external structures, for example with respect to any body to be treated with a plasma. The thickness of the layer or respectively the configuration of the structuring can, in this case, be selected as a function of the surface to be treated.

The advantage of this configuration is that a defined space for plasma production is provided or respectively a defined distance from the surface to be treated is guaranteed, which makes possible a plasma production or respectively a microbial treatment under defined conditions.

In one configuration, the spacer element is manufactured from a structured insulating, in particular electrically insulating, material.

The structure can, in this case, be e.g. a structured surface or can be configured by a textile, for example by a fabric, knitted fabric, mesh, stitch-bonded fabric, non-woven fabric and/or a felt.

A cover made of structured insulating material (textile fabric, perforated silicon mat, etc.) for observing a defined distance between the body tissue and the surgical instrument used as the plasma source can be utilized as the spacer element.

In another configuration, the device furthermore comprises the second electrode, wherein the second electrode is likewise arranged in a portion of the base body.

The second electrode is, as described, an earthed counter-electrode. It can be arranged circumferentially around the dielectric and/or the spacer element in the angular direction. Multiple portions of the base body can also have the second electrode or respectively portions of the second electrode.

In this configuration, the device for producing plasma is established without an external earthed counter-electrode. Consequently, an antimicrobial treatment can also be carried out in the region of non-conductive materials. The device according to the invention can consequently be used in a particularly versatile manner.

In another configuration, the second electrode is manufactured from electrically conducting material, in particular from a metallic fabric or a metal gauze.

The second electrode is consequently arranged in a portion of the base body, in particular in the same portion in which the high-voltage electrode is also arranged. It can be configured as a textile from a metallic material, e.g. from metallic fibers or fiber composites. All of the textiles described in connection with the spacer element can, in this case, be used individually or in combination.

Another cover from electrically conducting material (e.g. metal gauze) can, for example, be arranged above the spacer element, which cover is used instead of the body tissue as an earthed counter-electrode.

In addition to the advantages indicated above, the effectiveness in the moist medium is improved in the process, and the electromagnetic compatibility (EMC) guidelines can be observed.

In another configuration, the second electrode is arranged on the side of the dielectric facing away from the high-voltage electrode in at least one portion of the base body.

The second electrode can be arranged externally on the dielectric or—if a spacer element is present—externally thereon.

In another configuration of the device, a high-voltage-proof insulating layer is arranged between the base body and the high-voltage electrode.

This is in particular the case if the base body or respectively the outer surface thereof consists of an electrically conductive material so that the base body is in this way electrically insulated from the high-voltage electrode.

In particular, the insulating layer is likewise arranged circumferentially around the base body in the angular direction. It can, in this case, have a larger area than the high-voltage electrode and extends beyond this e.g. in the axial direction.

For example, in the case of a metallic outer tube of the base body, a high-voltage-proof insulating layer between the typically earthed metal pipe and the metal layer is additionally required.

This embodiment makes it possible to use objects having an electrically conducting base body as the device according to the invention.

In another configuration, the device is an instrument for endoscopy or laparoscopy, or a part of a catheter, or respectively a trocar or a canula.

The base body of the device according to the invention is therefore the base body or respectively shaft of an endoscope, laparoscope, trocar or catheter or a canula. Consequently, these instruments can be used simultaneously or alternatively to their original function as a plasma source and, indeed, prior to, during and/or after the known use for antimicrobial treatment of the body, in particular of the human or animal body. In this case, the edge regions of existing or made openings are in particular treated, in order to prevent an infection.

The shaft or respectively the base body can, in this case, be rigid or flexible. Therefore, it is likewise possible to equip a flexible element such as a hose by means of a suitable configuration and arrangement of the high-voltage electrode and, in particular, of the dielectric so that it can be used as a device for producing plasma according to the invention. Rigid base bodies are, for example, provided for instruments for minimally invasive surgery such as e.g. laparoscopes. Flexible base bodies are, for example, endoscopes or catheters. These can comprise metallic or non-metallic materials.

A device for performing a minimally invasive operation in accordance with the invention can be an instrument for endoscopy or laparoscopy as well as an appropriate part of a catheter (e.g. urinary catheters, vein catheters, arterial catheters, peridural catheters, port catheters, etc.), a trocar or a canula (for an arterial, venous, muscular, etc. access point). Such an instrument usually comprises a base body. According to the invention, this is a rigid or flexible, substantially elongated object which is suitable for being partially introduced into a body. In particular, it can be a rod, a bar, a hollow cylinder, a cable, a cable pull construction or a hose.

If the device for performing a minimally invasive operation according to the invention is designed as a trocar, it is a good idea if the tube used for guiding is produced from an electrically conductive material and is used as a counter-electrode.

An instrument, preferably for minimally invasive surgery, or respectively an endoscope is accordingly additionally used as a plasma-producing instrument according to the invention.

The device according to the invention can consequently be part of a medical apparatus for use in minimally invasive surgery. The advantage of this embodiment is that, as described, the known use can be supplemented by the antimicrobial treatment. Consequently, additional functionality is made possible, simply and without additional technical outlay

In particular, the base body of the device has a ratio of length to diameter of greater than 5:1 and, in particular, greater than 10:1.

The diameter is, in this case, measured at the thickest point of the base body. In other words, the base body is elongated. In this case, it can have a circular cross-section. In particular, it has a constant cross-section in one portion.

In another configuration, the high-voltage electrode and the dielectric and, in particular, the spacer element, the second electrode and/or the electrically insulating element are furthermore arranged in at least one portion of the base body in layers around the entire cross-section of the base body.

This means that they are arranged as layers, which can in particular each have a constant layer thickness, located above one another around the base body.

In particular, the high-voltage electrode, dielectric and, if applicable, further elements are arranged in the form of a ring and in layers around the base body. Consequently, the layers each run circumferentially around the base body in the angular direction. The advantage of this configuration is that the manufacture of the device is simple and that a plasma can be produced with this in the entire angular region around the base body, so that all of the circumferentially adjacent regions of the body can be antimicrobially treated.

A second aspect of the invention is a method for manufacturing a device for antimicrobial treatment according to the invention. Accordingly, the high-voltage electrode and the dielectric are applied to the surface of the base body using the thin-film method or using the thick-film method.

In particular, first the high-voltage electrode and then the dielectric are applied to the surface using one each of the indicated methods. Here, any method which can apply layers of conductive or respectively insulating material can be used. In this case, layers having the respectively desired properties are subsequently applied or respectively configured, wherein the respective layer thickness depends on the appropriate process parameters. This makes it possible to manufacture the device according to the invention simply and inexpensively.

For example, a high-voltage electrode for generating a dielectric barrier discharge (DBD) is applied to the surface of the base body of a minimally invasive instrument or respectively endoscope by a combined metallic and dielectrical coating using the thin-film or thick-film method, as a result of which the instrument itself, following the application of a suitable high voltage to the electrodes, can be used as a plasma source for an antimicrobially effective plasma treatment in the contact region with the body tissue, which can function as an earthed counter-electrode, in particular in the region of the edge of the wound at the inlet or respectively outlet opening.

A third aspect of the invention is a system for antimicrobial treatment during the performance of operations in bodies. Said system comprises a device for antimicrobial treatment according to the invention as well as a voltage source which is or can be connected electrically conductively to the device.

In this case, the voltage source is suitable for applying a voltage, with which a plasma, in particular a low-temperature plasma, can be produced by the device by means of the high-voltage electrode and an earthed counter-electrode.

In the case of an electrosurgical instrument, the medium-frequency or respectively high-frequency high voltage which is required for the function of the instrument anyway, can be applied during the treatment or when the instrument is extracted to the electrode applied to the base body, in order to achieve the described function of plasma production by the instrument itself. In this case, the instrument will not need its own voltage source.

An external voltage source or the available medium-frequency or respectively high-frequency high-voltage source can consequently be used for plasma production in the case of an electrosurgical instrument. Likewise, the available medium-frequency or respectively high-frequency high-voltage source can also be used, in the case of an electrosurgical instrument, as a voltage source for plasma production with an external plasma production apparatus (for example plasma source according to WO 2015 181 325 A1).

Thanks to the lower technical outlay, the handling is made substantially easier and, as a result, a significant time saving is achieved.

In one configuration of the system, the latter comprises the body which is to be treated with a plasma at least in certain areas, wherein at least a part of the body configures the second electrode.

In other words, a device is provided, wherein the second electrode is arranged on or in the body, in which the operation is to be performed, or is configured by the body. Consequently, the body to be examined itself functions as an earthed counter-electrode. Accordingly, the system for performing a minimally invasive operation also has the body itself.

In particular, the device does not itself comprise the second electrode in this configuration.

A fourth aspect of the invention is a method for antimicrobial treatment during the performance of operations in bodies, in which the device for antimicrobial treatment according to the invention is introduced into the body. A plasma is produced by means of the device at least in the region of the opening of the body, through which the device for performing a minimally invasive operation is introduced into the body. The plasma is typically brought into contact with the surface to be treated in order to perform the antimicrobial treatment.

The body can comprise human and/or animal tissue. Independently thereof, the method can be performed on the human or animal body or outside the human or animal body.

The production of the plasma is in particular used, as described, for the antimicrobial treatment of the appropriate region of the body. In particular, the device is partially introduced into the body, for example with its base body. In this case, an existing opening can be used, for example in the case of a catheter, or an opening can be produced, for example in the case of a canula or a trocar.

The invention furthermore comprises a method for antimicrobial treatment during the performance of operations in bodies, not for antimicrobial treatment of human or animal tissue on the human or animal body, in which the device for antimicrobial treatment according to the invention is introduced into the body. A plasma is produced by means of the device at least in the region of the opening of the body, through which the device for performing a minimally invasive operation is introduced into the body. The plasma is typically brought into contact with the surface to be treated in order to perform the antimicrobial treatment.

The method according to the invention can be actively monitored by means of electronics and software for controlling uniform and safe treatment.

A fifth aspect of the invention is a computer program which carries out all of the steps in order to perform the method according to the invention for antimicrobial treatment when the program runs on a computer.

The computer program can, in this case, control the plasma production: for example, it can start this, end this, shut down and/or monitor a defined program. It can furthermore be set up to process and use measured values.

In particular, this can be a computer program which carries out all of the steps in order to perform a method for antimicrobial treatment during the performance of operations in bodies when the program runs on a computer. In the case of the indicated method, the device for antimicrobial treatment according to the invention is introduced into the body and a plasma is produced by means of the device, at least in the region of the opening of the body, through which the device for performing a minimally invasive operation is introduced into the body. Alternatively, the computer program can be one which carries out all of the steps in order to perform a method for antimicrobial treatment during the performance of operations in bodies, not for antimicrobial treatment of human or animal tissue on the human or animal body when the program runs on a computer. In the case of the indicated method, the device for antimicrobial treatment according to the invention is introduced into the body and a plasma is produced by means of the device at least in the region of the opening of the body, through which the device for performing a minimally invasive operation is introduced into the body.

Another aspect of the invention is a computer program product having program code stored on a machine-readable carrier in order to perform the method according to the invention when the program is run by a computer, in particular by a microcontroller integrated into an electronic control instrument.

The subject matter of the invention is likewise a computer program product, having a program code of the computer program according to the invention stored on a machine-readable carrier. Machine-readable carriers can be all data carriers, in particular all digital data carriers.

The invention will be explained below with reference to the embodiment example which is represented in the appended drawings, wherein:

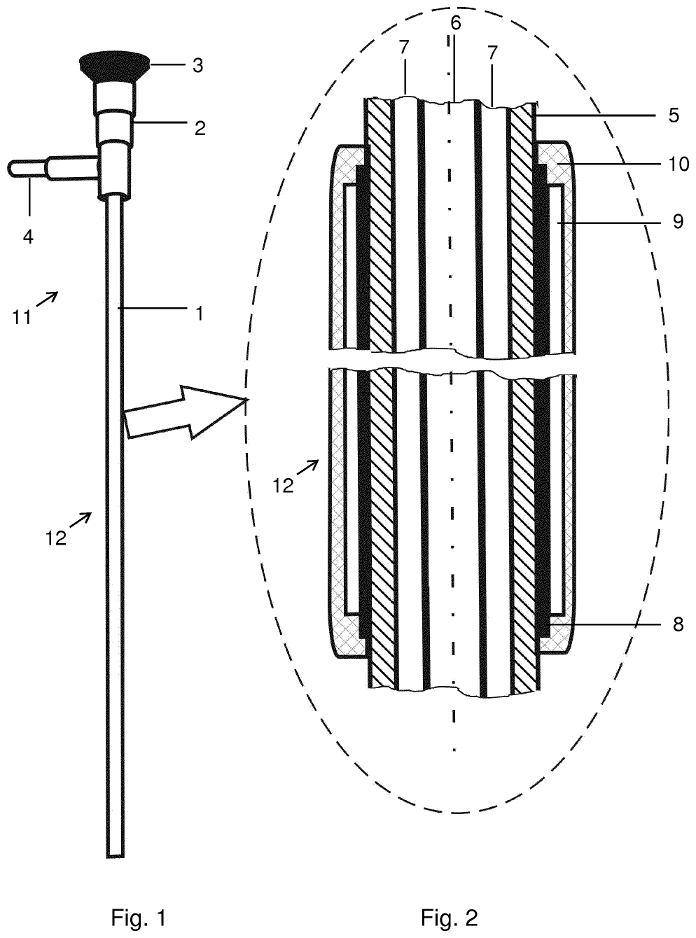

FIG. 1 shows a perspective representation of a device according to the invention, designed as a laparoscope, as well as

FIG. 2: shows a cutaway representation of a detail from the base body of the device from FIG. 1.

FIG. 1 shows the device 11 according to the invention, designed as a laparoscope. This is a simple instrument for minimally invasive surgery and comprises an elongated base body 1 which is set up for at least partial introduction into a body, in particular a human or animal body. A handpiece 2 having an eyepiece 3 and a lateral light guide connector 4 is arranged on the side of the base body which is shown at the top and which is opposite the side to be introduced into the body.

Joined to the light guide connector 4 in the interior of the base body 1 is a light channel 7, in which light can be conducted from an external source of light which can be joined to the light guide connector 4 into the interior of the body to be examined. The light channel 7 has a circular shape and encloses the optical channel 6 located therein, which is set up to conduct the light radiation reflected by surfaces in the interior of the body towards the eyepiece 3. Consequently, optical information can be conducted from the interior of the body to the user of the device. The optically conducting channels 6, 7 indicated are visible in the sectional drawing shown in FIG. 2.

The base body 1 is designed as a metal pipe having a circular cross-section, in which fixtures are arranged for the purposes of subdividing the indicated optically conducting channels 6, 7.

According to the invention, the outer tube 5 of the base body 1 is coated with multiple layers all round in a partial region of the length in a defined way, in order to be able to use this region as a high-voltage electrode 9 for producing a plasma. In order to demonstrate the fundamental layer construction which is required for this, a part of the coated base body 1 is represented in an enlarged sectional drawing in FIG. 2.

A high-voltage-proof insulating layer 8 is arranged on the metallic outer tube 5 of the base body 1, which insulating layer electrically insulates the base body 1, which is earthed, from the high-voltage electrode 9 arranged around this. Consequently, an electrical potential difference which is necessary for producing the plasma is made possible between the base body 1 and the high-voltage electrode 9.

The high-voltage electrode 9 for producing a dielectric barrier discharge is formed by a metal layer covered with a dielectric layer 10. The dielectric 10 projects, in this case, beyond the high-voltage electrode 9 on both sides in the axial direction, therefore extends over the upper and below the lower end of the high-voltage electrode 9, wherein it contacts the insulating layer 8 in these regions and, extending beyond these, rests on the metallic base body 1. Consequently, the dielectric 10 serves as a sheathing or respectively cover which protects the described layers, for example against moisture.

The described layers are arranged above the majority of the region of the base body 1 which can be introduced into the body.

Depending on the quality of the base bodies 1 of the minimally invasive instruments or endoscopes (surface, form, size and material type), the invention can be technically implemented in different ways, resulting in the realization of various variants.

As a rule, this principle can be applied to all instruments which are introduced in the form of a rod, endoscope or catheter transcutaneously or percutaneously into the body. This is equally true of surgical instruments, such as e.g. laparoscopes, and endoscopes. In this case, an external power supply is merely required for the plasma production.

### LIST OF REFERENCE NUMERALS

1 Base body
2 Handpiece
3 Eyepiece
4 Light guide connector
5 Outer tube of the base body
6 Optical channel
7 Light channel
8 Insulating layer
9 High-voltage electrode (metal layer)
10 Dielectric (dielectric layer)
11 Device
12 Plasma source

1. A device (11) for antimicrobial treatment during the performance of operations, in particular minimally invasive operations, in bodies, having a base body (1) for partial introduction into a body,
characterized in that the device (11) furthermore comprises at least one plasma source (12) arranged in at least one portion of the base body (1), wherein the plasma source (12) has at least one high-voltage electrode (9) which is at least partially covered and in particular completely covered with a dielectric (10), which high-voltage electrode is set up to produce a plasma by means of dielectric barrier discharge when an electrical voltage is applied and in conjunction with a second electrode.

2. The device (11) for antimicrobial treatment according to claim 1, characterized in that a spacer element for producing a distance between the plasma source (12) and body tissue is arranged in at least one portion of the base body (1) on the side of the dielectric (10) facing away from the high-voltage electrode (9).

3. The device (11) for antimicrobial treatment according to claim 2, characterized in that the spacer element is manufactured from a structured insulating, in particular electrically insulating, material.

4. The device (11) for antimicrobial treatment according to claim 1, characterized in that said device furthermore comprises the second electrode, wherein the second electrode is likewise arranged in a portion of the base body (1).

5. The device (11) for antimicrobial treatment according to claim 4, characterized in that the second electrode is manufactured from electrically conducting material, in particular a metallic fabric or a metal gauze.

6. The device (11) for antimicrobial treatment according to claim 4, characterized in that the second electrode on the side of the dielectric (10) facing away from the high-voltage electrode (9) is arranged in at least one portion of the base body (1).

7. The device (11) for antimicrobial treatment according to claim 1, characterized in that a high-voltage-proof insulating layer (8) is arranged between the base body (1) and the high-voltage electrode (9).

8. The device (11) for antimicrobial treatment according to claim 1, characterized in that the device (11) is an instrument for endoscopy, laparoscopy, or a part of a catheter, or a trocar or a canula.

9. The device (11) for antimicrobial treatment according to claim 1, characterized in that the high-voltage electrode (9) and the dielectric (10) and, in particular, the spacer element, the second electrode and/or the electrically insulating element are furthermore arranged in at least one portion of the base body (1) in layers around the entire cross-section of the base body (1).

10. A method for manufacturing a device (11) for antimicrobial treatment according to claim 1, characterized in that the high-voltage electrode (9) and the dielectric (10) are applied to the surface of the base body (1) using the thin-film process or using the thick-film process.

11. A system for antimicrobial treatment during the performance of operations in bodies, characterized in that the system comprises a device (11) for antimicrobial treatment according to claim 1 as well as a voltage source which is or can be connected electrically conductively to the device (11).

12. The system for antimicrobial treatment according to claim 11, characterized in that the system comprises the body which is to be treated at least in certain areas with a plasma, wherein at least one part of the body forms the second electrode.

13. A method for antimicrobial treatment during the performance of operations in bodies, in which the device (11) for antimicrobial treatment according to claim 1 is introduced into the body and a plasma is produced by means of the device (11) at least in the region of the opening of the body, through which the device (11) for performing a minimally invasive operation is introduced into the body.

14. A method for antimicrobial treatment during the performance of operations in bodies, not for antimicrobial treatment of human or animal tissue on the human or animal body, in which the device (11) for antimicrobial treatment according to claim 1 is introduced into the body and a plasma is produced by means of the device (11) at least in the region of the opening of the body, through which the device (11) for performing a minimally invasive operation is introduced into the body.

15. A computer program which carries out all of the steps for performing the method for antimicrobial treatment according to claim 13 when the program runs on a computer.
